(19) United States
(12) Patent Application Publication
(10) Pub. No.: US 2010/0273848 A1
(43) Pub. Date: Oct. 28, 2010

(54) IMINOPYRROLIDONE THIOL AMINO ACID CONJUGATES, PHARMACEUTICAL COMPOSITIONS AND METHODS FOR THE TREATMENT OF CANCER

(75) Inventors: Ross O. Meyers, Oregon, WI (US); Robert T. Dorr, Tucson, AZ (US)

Correspondence Address: OBLON, SPIVAK, MCCLELLAND MAIER & NEUSTADT, L.L.P., 1940 DUKE STREET, ALEXANDRIA, VA, 22314, US

(73) Assignee: THE ARIZONA BD OR REG ON BEHALF OF THE UNIV OF AZ, Tucson, AZ (US)

(21) Appl. No.: 12/593,143

(22) PCT Filed: Mar. 14, 2008
(86) PCT No.: PCT/US08/56925

§ 371 Date: Jun. 15, 2010

Related U.S. Application Data
(60) Provisional application No. 60/920,005, filed on Mar. 26, 2007.

(51) Int. Cl. A61K 31/4166; C07D 233/32; A61P 35/00; A61P 35/02

(52) U.S. Cl. 514/389; 548/321.5

(57) ABSTRACT

Compounds, compositions and methods for the treatment of cancer are disclosed herein. Embodiments of the present invention include compounds having the formula: (I), wherein R1 is selected from hydrogen or substituted or unsubstituted (C₁-C₆) alkyl or (C₃-C₆) cycloalkyl, R² and R³ are independently selected from hydrogen, substituted or unsubstituted (C₁-C₆) alkyl, (C₄-C₆) cycloalkyl, or taken together with the N atom to which they are attached form a 3-6 membered heterocycloalkyl, and n is 1-5. Embodiments of the present invention also include pharmaceutical compositions comprising compounds of Formula (I) or pharmaceutically acceptable salts thereof, and methods of treating cancer via the administration of such compounds to patients in need of such treatment.

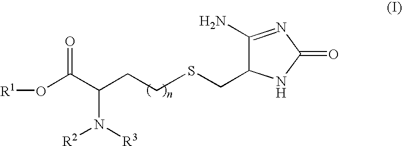

### CROSS-REFERENCE TO RELATED APPLICATIONS

This application claims priority to U.S. provisional patent application No. 60/920,005, filed Mar. 26, 2007, the disclosure of which is incorporated herein by reference in its entirety for all purposes.

### STATEMENT REGARDING FEDERALLY SPONSORED RESEARCH

This invention was made with government support under CA-23074 awarded by the National Cancer Institute, National Institutes of Health. The U.S. government has certain rights in the invention.

### FIELD OF THE INVENTION

This disclosure relates generally to iminopyrrolidone thiol amino acid conjugates, and more particularly, but not exclusively, to iminopyrrolidone thiol amino acid conjugate compounds and pharmaceutical compositions, and to methods utilizing iminopyrrolidone thiol amino acid conjugates in the treatment of cancer.

### BACKGROUND INFORMATION

Cancer is a leading cause of death among humans and animals. As such, considerable resources are invested in the identification and development of compounds and compositions useful in treating the many forms of the disease. While numerous therapeutic options have been developed through extensive research efforts, and have, in many cases, led to dramatic improvements in the treatment and survival of cancer patients, continued progress in treating the many forms of the disease requires the development of additional therapeutic options. Unfortunately, in many instances, not all patients respond to existing therapeutic agents, and patients frequently develop resistance to current therapies as treatment proceeds. Because of these disadvantages, new therapeutic options that may overcome such resistance or provide an effective therapy for another cancer patient population are desirable.

### SUMMARY OF THE INVENTION

The present invention is directed to iminopyrrolidone amino acid conjugates and pharmaceutically acceptable salts thereof having anticancer activity, to pharmaceutical compositions comprising iminopyrrolidone amino acid conjugates, to methods of treating cancer via the administration of iminopyrrolidone amino acid conjugates to patients in need thereof, and to pharmaceutical kits comprising iminopyrrolidone amino acid conjugates and instructions for their use in the treatment of cancer.

In one aspect, the present invention provides a compound having the formula:

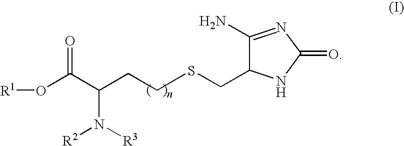

In Formula (I), R¹ is selected from hydrogen or substituted or unsubstituted (C₁-C₆) alkyl or (C₃-C₆) cycloalkyl. R² and R³ are independently selected from hydrogen, substituted or unsubstituted (C₁-C₆) alkyl, (C₄-C₆) cycloalkyl, or taken together with the N atom to which they are attached form a 3-6 membered heterocycloalkyl, and n is 1-5.

In another aspect, the present invention provides a pharmaceutical composition comprising a unit dose of a compound of Formula (I), wherein R¹, R², R³, and n are as defined hereinbefore, and a pharmaceutically acceptable carrier.

In another aspect, the present invention provides a method of treating cancer, comprising administering to a patient in need thereof a therapeutically effective amount of a composition comprising a compound of Formula (I), wherein R¹, R², R³, and n are as defined hereinbefore.

In yet another aspect, the present invention provides a pharmaceutically acceptable salt of a compound of Formula (I), wherein R¹, R², R³, and n are as defined hereinbefore.

In still another aspect, the present invention provides a pharmaceutical kit comprising a compound of Formula (I), wherein R¹, R², R³, and n are as defined hereinbefore, and printed instructions for using the compound in the treatment of cancer.

### BRIEF DESCRIPTION OF THE DRAWINGS

FIG. 1 is a graphical illustration of the cytotoxic tumor inhibitory effects of simexonyl homocysteine in A375 human malignant melanoma cells in vitro.

FIG. 2 is a graphical illustration of the cytotoxic tumor inhibitory effects of simexonyl homocysteine in PC-3 human prostate cancer cells in vitro.

FIG. 3 is a graphical illustration of the cytotoxic tumor inhibitory effects of simexonyl homocysteine in DU-145 human prostate cancer cells in vitro.

FIG. 4 is a graphical illustration of the cytotoxic tumor inhibitory effects of simexonyl homocysteine in chemotherapy sensitive (8226/s) and chemotherapy resistant (8226/110) human multiple myeloma cells in vitro.

FIG. 5 is a graphical illustration comparing the cytotoxic tumor inhibitory effects of simexonyl homocysteine with the cytotoxic tumor inhibitory effects of a chemotherapeutic shown to have anticancer activity in clinical studies (imexon) in MiaPaCa human pancreatic cancer cells in vitro.

FIG. 6 is a graphical illustration of the results of a series of apoptosis assays for simexonyl homocysteine in DB lymphoma cells.

### DETAILED DESCRIPTION OF EMBODIMENTS OF THE INVENTION

Embodiments of compounds of Formula (I), pharmaceutical compositions and methods for the treatment of cancer are disclosed herein. In the following description, numerous specific details are provided, such as the identification of various components and structures, to provide a thorough understanding of embodiments of the invention. One skilled in the art will recognize however, that the invention can be practiced without one or more of the specific details, or with other methods, components, materials, and the like. In still other instances, well-known components, materials, or processes are not shown or described in detail to avoid obscuring aspects of various embodiments of the invention.

Reference throughout this specification to “one embodiment” or “an embodiment” means that a particular feature, structure, component, or characteristic described in connection with the embodiment is included in at least one embodiment of the present invention. Thus, the appearance of the phrases “in one embodiment” or “in an embodiment” in various places throughout this specification are not necessarily all referring to the same embodiment. Furthermore, the particular features, structures, components, or characteristics may be combined in any suitable manner in one or more embodiments.

### I. DEFINITIONS

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “therapeutically effective amount” refers to the amount of a compound or composition effective to yield the desired therapeutic response. The therapeutically effective amount may vary with such factors as the particular condition or clinical pattern of disease being treated, the physical condition of the patient, the duration of the treatment, the nature of concurrent therapy (if any), and the specific formulations employed.

The term “alkyl,” by itself or as part of another substituent, means, unless otherwise stated, a straight (i.e. unbranched) or branched chain hydrocarbon radical which may be fully saturated, mono- or polyunsaturated and can include di- and multivalent radicals, having the number of carbon atoms designated (e.g. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Similarly, the term “alkylene” by itself or as part of another substituent means a divalent radical derived from alkyl, as exemplified, but not limited by, methylene (—CH₂—), ethylene (—CH₂—CH₂—), propylene (—CH₂—CH₂—CH₂—), and isopropylene (—CH₂(CH₃)—CH₂—).

The term “heteroalkyl,” by itself or in combination with another term, means, unless otherwise stated, a stable straight or branched chain hydrocarbon radical consisting of at least one carbon atom and at least one heteroatom selected from the group consisting of O, N and S, and wherein the nitrogen and sulfur atoms may optionally be oxidized and the nitrogen heteroatom may optionally be quaternized. The heteroatom(s) O, N and S may be placed at any interior position of the heteroalkyl group or at the position at which the heteroalkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —CH₂—CH═N—OCH₃, —CH═CH—N(CH₃)—CH₃, —O—CH₃, —O—CH₂—CH₃, —NH—CH₂—OH, —CH(OH)—CH₃, —C(O)—CH₂—OH, —C(O)—CH₂—O—C(O)—CH₂—CH₃, —O—C(O)—C(CH)₃, and —O—C(O)—CH₂—CH₃. Up to three heteroatoms may be consecutive, such as, for example, —CH₂—NH—OCH₃ and —N═N—N(CH₃)₂. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopropylmethyl, cyclopentyl, cyclohexyl, cyclohexylmethyl, 1-cyclohexenyl, 3-cyclohexenyl, cycloheptyl, and the like. Examples of heterocycloalkyl include, but are not limited to, 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl 1-pyrrolidinyl, 2-pyrrolidinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. The terms “arylene” and “heteroarylene” refer to the divalent derivatives of aryl and heteroaryl, respectively.

For brevity, the term “aryl” when used in combination with other terms (e.g., aryloxo, arylthioxo, and arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 341-naphthyloxy)propyl, and the like). However, the term “haloaryl,” as used herein, is meant to cover only aryls substituted with one or more halogens.

The term “oxo” means an oxygen atom that is double bonded to a carbon atom.

Each of above terms (e.g., “alkyl,” “heteroalkyl,” “cycloalkyl,” “heterocycloalkyl,” “aryl,” “heteroaryl,” and “arylalkyl,” as well as their divalent radical derivatives) are meant to include both substituted and unsubstituted forms of the indicated radical. Preferred substituents for each type of radical are provided below.

Substituents for alkyl, heteroalkyl, cycloalkyl, heterocycloalkyl monovalent and divalent derivative radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —OC(O)R¹, —C(O)R¹, —CO₂R¹, —C(O)NR′R″, —OC(O)NR′R″, —NR″C(O)R¹, —NR′—C(O)NR″R′″, —NR″C(O)OR′, —NR—C(NR′R″)═NR′″, —S(O)R¹, —S(O)₂R¹, —S(O)₂NR′R″, —NRSO₂R¹, —CN and —NO₂ in a number ranging from zero to (2 m′+1), where m′ is the total number of carbon atoms in such radical. R′, R″ and R′″ each preferably independently refer to hydrogen, or C₁-C₆ alkyl, cycloalkyl, or haloalkyl. Unless otherwise stated, when a compound of the invention includes more than one R group, each of the R groups is independently selected as are each R′, R″ and R′″ groups when more than one of these groups is present.

Similar to the substituents described for alkyl radicals above, exemplary substituents for aryl and heteroaryl groups (as well as their divalent derivatives) are varied and are selected from, for example: —OR′, —NR′R″, —SR′, -halogen, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR′″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on the aromatic ring system; and where R′, R″, R′″ and R′″ are preferably independently selected from hydrogen, or C₁-C₆ alkyl, cycloalkyl, or haloalkyl. Unless otherwise stated, when a compound of the invention includes more than one R group, each of the R groups is independently selected as are each R′, R″, R′″ and R′″ groups when more than one of these groups is present.

As used herein, the term “heteroatom” or “ring heteroatom” is meant to include oxygen (O), nitrogen (N), and sulfur (S).

The compounds of the present invention may exist as salts. The present invention includes such salts. These salts may be prepared by methods known to those skilled in art. The term “pharmaceutically acceptable salts” is meant to include salts of active compounds which are prepared with relatively nontoxic acids or bases, depending on the particular substituent moieties found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The terms “a,” “an,” or “a(n)”, when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

### II. IMINOPYRROLIDONE THIOL AMINO ACID CONJUGATES OF THE PRESENT INVENTION

Embodiments of the present invention include iminopyrrolidone thiol amino acid conjugate compounds useful for the treatment of cancer, including carcinomas, sarcomas, melanomas, leukemias, and lymphomas. Compounds in accordance with the present invention have the formula:

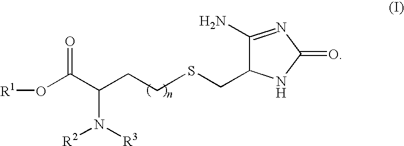

In Formula (I), R¹ is selected from hydrogen or substituted or unsubstituted (C₁-C₆) alkyl or (C₃-C₆) cycloalkyl, R² and R³ are independently selected from hydrogen, substituted or unsubstituted (C₁-C₆) alkyl, (C₄-C₆) cycloalkyl, or taken together with the N atom to which they are attached form a 3-6 membered heterocycloalkyl, and n is 1-5. It will be appreciated that certain compounds of the present invention may exist in tautomeric forms in which two or more structural isomers exist in equilibrium and are readily converted from one isomeric form to another. All such tautomeric forms of the compounds are intended to come within the scope of the present invention. It will further be appreciated that compounds of the present invention include one or more chiral centers that define enantiomers or other stereoisomers of the iminopyrrolidone thiol amino acid conjugates. All such stereoisomers shall, unless otherwise indicated, be encompassed by the formulae and descriptions provided herein and are intended to come within the scope of the present invention whether the compounds exist as racemic mixtures or as steroisomerically-enriched or pure compositions.

In one embodiment, compounds of the present invention comprise those compounds having a structure consistent with Formula (I), wherein R¹, R², and R³ are as defined hereinbefore with reference to Formula (I), and n is 1-3. In another embodiment, compounds of the present invention comprise those compounds having a structure consistent with Formula (I), wherein R² and R³ are as defined hereinbefore with reference to Formula (I), R¹ is hydrogen, and n is 1. In yet another embodiment, compounds of the present invention comprise those compounds having a structure consistent with Formula (I), wherein R¹ is as defined hereinbefore with reference to Formula (I), R² and R³ are hydrogen, and n is 1. In still another embodiment, compounds of the present invention comprise those compounds having a structure consistent with Formula (I), wherein R², R³, and n are as defined hereinbefore with reference to Formula (I), and R¹ is (C₁-C₃) alkyl. In a preferred embodiment, R¹, R², and R³ are hydrogen, and x is 1.

A. Methods of Synthesizing Iminopyrrolidone Thiol Amino Acid Conjugates

Iminopyrrolidone thiol amino acid conjugates of the present invention can be prepared according to the synthesis schemes outlined hereinafter, and may employ laboratory techniques generally apparent and accessible to those of skill in the relevant art. In Schemes I-IV, R¹, R², R³ and n are as defined previously with reference to Formula (I), and all other variables are as defined with reference to the illustrated schemes.

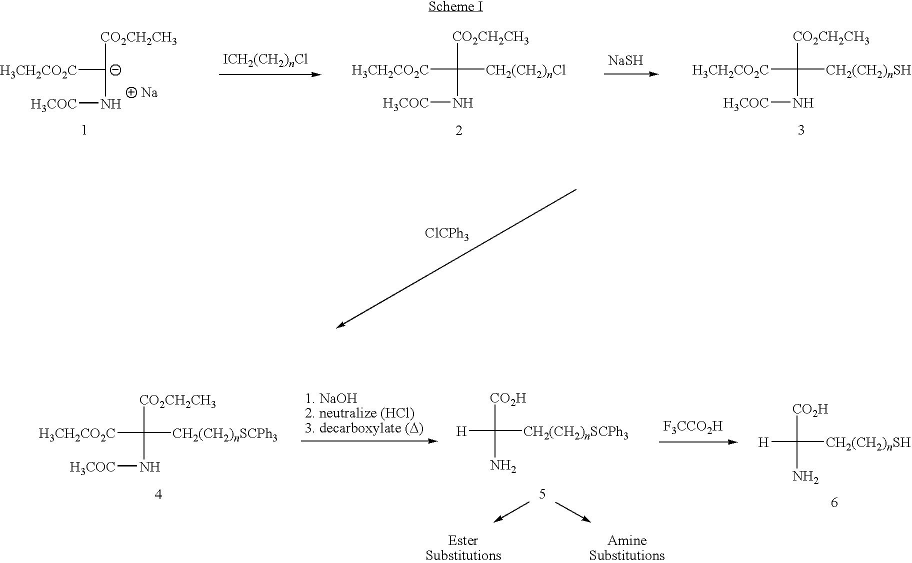

In Scheme (I), an amino acid functionality is coupled to a variable length chain of methylene groups by reacting the sodium or potassium salt of diethyl N-acetylaminomalonic ester 1 with a chloro-alkyliodide having the structure ICH₂(CH₂)ₙCl, whereby displacement of the iodine atom yields the intermediate compound 2. As will be appreciated, the chloro-alkyliodide may comprise 1-chloro-2-iodoethane, 1-chloro-3-iodopropane, 1-chloro-4-iodobutane, 1-chloro-5-iodopentane, or 1-chloro-6-iodohexane to yield a compound in which n is 1-5. Displacement of the chlorine atom from intermediate compound 2 with sodium hydrogen sulfide yields the thiol compound 3 having the desired chain of methylene groups connecting the amino acid functionality and the sulfhydryl moiety. The thiol moiety of 3 is then protected by, for example, a triphenylmethyl(trityl) group to yield a protected compound 4, and the ethyl esters are then hydrolyzed by base and the resulting dicarboxylic acid is decarboxylated to give the trityl-protected thiol amino acid 5.

The trityl-protected thiol amino acid 5 can be deprotected by treatment with trifluoroacetic acid to yield the thiol amino acid 6. Alternatively, the trityl protected thiol amino acid 5 may undergo ester or amine substitutions to generate the various R¹, R², and R³ substituents as defined previously with reference to Formula (I), and as will be described and illustrated in greater detail hereinbelow.

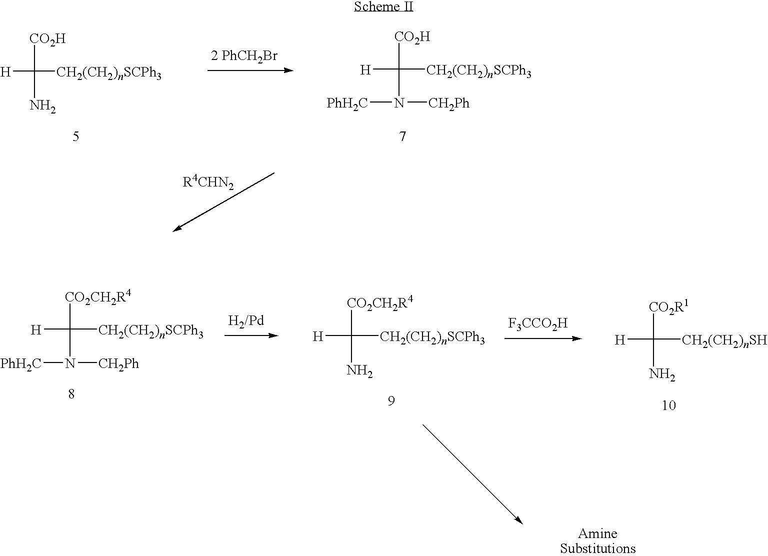

In Scheme (II), ester substitutions of the trityl-protected thiol amino acid 5 are carried out by first protecting the amino group with an amino-protecting group, such as for example benzyl bromide, to yield the di-protected thiol amino acid 7. The addition of the ester group can then be achieved by reacting the di-protected thiol amino acid 7 with an appropriate diazoalkane having the structure R⁴CHN₂, in which R⁴ is hydrogen, alkyl, or cycloalkyl, to give the ester 8. The amino protecting groups can then be removed via hydrogenation to yield the ester-substituted trityl-protected thiol amino acid 9, which can be deprotected by treatment with trifluoroacetic acid, as described previously in conjunction with Scheme (1), to yield the ester-substituted thiol amino acid 10. As will be appreciated, where R⁴ is hydrogen in 9, R¹ will be methyl in 10, where R⁴ is methyl in 9, R¹ will be ethyl in 10, and so on. Alternatively, the ester-substituted trityl-protected thiol amino acid 9 can undergo amine substitutions as will be described hereinbelow.

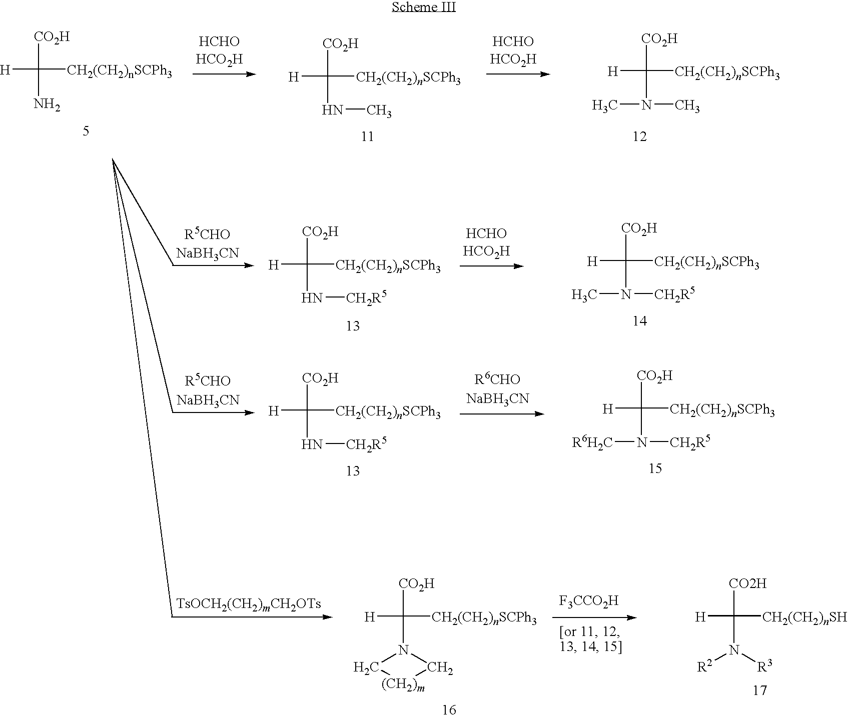

In Scheme (III), amine substitutions of the trityl-protected thiol amino acid 5 can be carried out via a variety of different synthetic pathways to yield the desired end product. Although not illustrated, it will be appreciated that the ester-substituted trityl-protected thiol amino acid 9 (from Scheme (II)) can comprise the starting material in each of the synthetic pathways illustrated in Scheme (III) to yield a compound having both ester and amine substitutions. In one pathway, the trityl-protected thiol amino acid 5 can be methyl substituted to give the methylamino compound 11 through reaction with formaldehyde and formic acid. Further reaction of these same components can yield the dimethylamino thiol amino acid 12. Where at least one of the desired amine substitutions comprises a larger alkyl substituent, the appropriate trityl-protected thiol amino acid 5 can be contacted with the appropriate aldehyde (e.g., acetaldehyde, propionaldehyde, butyraldehyde, etc.) in the presence of sodium cyanoborohydride to yield the mono-substituted compound 13. The mono-substituted compound 13 can in turn be substituted a second time by either of the approaches previously described to give the disubstituted compounds 14 or 15. In the synthetic pathways described and illustrated in Scheme (III), R⁵ and R⁶ can be the same or different, and can comprise alkyl, cycloalkyl, or aryl.

In an alternative synthetic pathway illustrated in Scheme (III), the trityl-protected thiol amino acid 5 can be substituted to yield a heterocyclic compound 16 by reaction with an alkane ditosylate having the formula TsO—CH₂—(CH₂)ₘ—CH₂—OTs in which m is 0-3. Each of the amine substituted compounds 11, 12, 13, 14, 15, 16 can then be deprotected by treatment with trifluoroacetic acid, as described previously in conjunction with Scheme (I), to yield the amine-substituted thiol amino acid 17. As will be appreciated, where R⁵ or R⁶ is methyl in 13, 14, or 15, R² or R³ will be ethyl, respectively, in 17, and so on.

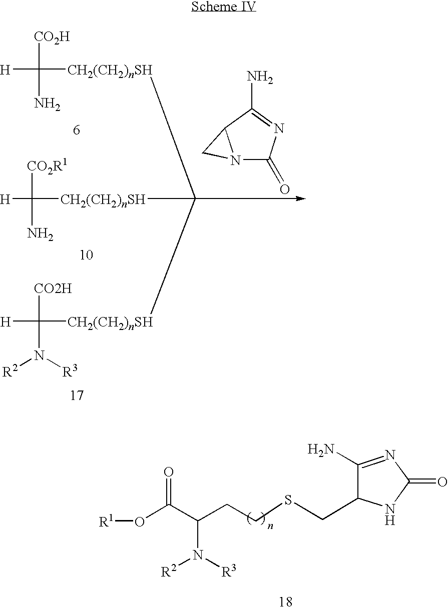

In Scheme (IV), any one of the deprotected thiol amino acid compounds 6, 10, or 17 can be converted to an iminopyrrolidone thiol amino acid conjugate 18 in accordance with the present invention, in which R¹, R², R³, and n are as defined with reference to Formula (I), by contacting the thiol amino acid with one equivalent of (5RS)-4-amino-1,3-diazabicyclo[3.1.0]hex-3-en-2-one (imexon). In one embodiment, the reaction may be carried out in an aqueous environment at pH 7.5 at a temperature of 37° C., wherein the reaction is monitored by HPLC and stopped by freezing at −20° C. when the concentration of the product is maximized. For example, where the thiol amino acid is homocysteine, the reaction time is approximately 5 hours. As will be appreciated, the thiol amino acids 6, 10, and 17, illustrated in Scheme (IV), represent examples from the prior schema and are intended to encompass the unillustrated combination with both ester and amine substitutions or any combination which yields the iminopyrrolidone thiol amino acid conjugate 18 with the various substituents R¹, R², R³, and n as defined with reference to Formula (I).

In an alternate embodiment, the synthesis of a preferred compound in which R¹, R², and R³ are hydrogen and n is 1 may be carried out by converting homocysteine thiolactone to homocysteine, followed by conversion to the iminopyrrolidone thiol amino acid conjugate 18, as described in reference to Scheme (IV). This alternative synthesis route is described in greater detail in the Examples.

As described hereinbefore, compounds of the present invention may exist as pharmaceutically acceptable salts, prepared by known methods, and including without limitation, chlorides, phosphates, sulfates, tosylates, benzoylates and mesylates.

B. Assays for Testing the Anticancer Activity of Iminopyrrolidone Thiol Amino Acid Conjugates

Compounds of the present invention are useful as antineoplastic agents. A number of biological assays for testing the antineoplastic activity of iminopyrrolidone thiol amino acid conjugates of the present invention are available. These assays can roughly be split into two groups: Those involving in vitro exposure of tumor cells to a selected agent; and in vivo antitumor assays in rodent models, and rarely, in larger animals. Both types of assays are equally applicable to determining whether an iminopyrrolidone thiol amino acid conjugate of the present invention exhibits antineoplastic activity. As used herein, the term “antineoplastic” means inhibiting or preventing the growth of cancer, including reducing the growth of cancer relative to the absence of a given therapy or treatment.

In vitro cytotoxic assays generally involve the use of established tumor cell lines both of animal, and, especially, of human origin. These cell lines can be obtained from commercial sources such as the American Type Tissue Culture laboratory in Manassas, Va., and from tumor banks at research institutions. Exemplary cell line models representing specific cancers which may be used to evaluate the susceptibility of a particular tumor type to the compounds of the present invention via the methods described hereinafter and in the indicated references include, but are not limited to, the following human tumor cell lines.

Cervical cancer: HeLa; HeLa 229; HeLa S3; H1HeLa; Hs 588.T; GH329; GH354; HeLa NR1; C-4 I; C-4 II; DoTc2 4510; C-33 A; SW756; SiHa.

Colorectal cancer: NCI-H548; Hs 255.T; SW837; NCI-H716; NCI-H747; NCl—HSO8; NCI-H498; WiDr; COLO 320DM; COLO 320HSR; DLD-1; HCT-15; SW480; SW403; SW48; SW1116; SW948; SW1417; LS123; LS180; LS174T; C2BBe1; Hs 257.T; Hs 587.Int; Caco-2; HT-29; SW1463; Hs 200.T; Hs 219.T; Hs 675.T; SNU-C2B; SNU-C2A; LS513; LS1034; LS411N; HCT 116; ATRFLOX; Hs 722.T.

Kidney cancer: A704; A-704; ACHN; 786-O; 769-P; A-498; Caki-2; G-402; Hs 926.T; G-401.

Lung cancer: NCI-H1373; NCI-H1395; Hs 618.T; SK-LU-1; HCC2935; HCC4006; HCC827; Hs 229.T; NCI-H2066; NCI-H2286; Hs 573.T; A549; A-427; NCI-H596; NCI-H292; DMS 79; DMS 53; DMS114; SW 1271; NCI-H2227; NCI-H1963; SHP-77; H69AR; NCI-H2170; NCI-H520; SW 900.

Non small-cell lung cancer: NCI-H1581; NCI-H23; NCI-H522; NCI-H1435; NCl-H1563; NCI-H1651; NCI-H1734; NCI-H1793; NCI-H1838; NCI-H1975; NCI-H2073; NCl-H2085; NCI-H2228; NCI-H2342; NCI-H2347; NCI-H1703; NCI-H2135; NCI-H2172; NCl-H2444; NCI-H358; NCI-H1688; NCI-H1417; NCI-H1672; NCI-H1836; PLHC-1; NCl-H810; NCI-H2126.

Breast cancer: Hs 274.T; Hs 280.T; Hs 281.T; Hs 343.T; Hs 362.T; Hs 739.T; Hs 741.T; Hs 742.T; Hs 190.T; Hs 319.T; Hs 329.T; Hs 344.T; Hs 350.T; Hs 371.T; Hs 748.T; Hs 841.1°; Hs 849.T; Hs 851.T; Hs 861.T; Hs 905.T; Hs 479.T; Hs 540.T; Hs 566(B).T; Hs 605.T; Hs 606; BT-20; UACC-812; HCC1954; Hs 574.T; BT-483; BT-549; DU4475; Hs 578T; BT-474; HCC1806; HCC38; UACC-893; HCC70; HCC202; HCC1143; HCC1187; HCC1395; HCC1419; HCC1500; HCC1599; HCC1937; HCC2157; HCC2218; HCC1569.

Pancreas cancer: BxPC-3; HPAF-II; HPAC; Panc 03.27; Panc 08.13; Panc 02.03; Panc 02.13; Pane 04.03; Panc 05.04; Capan-2; CFPAC-1; PL45; Panc 10.05; MIA PaCa-2; PANC-1.

Testicular cancer: Cates-1B.

Ovarian cancer: Caov-3; TOV-21G; TOV-112D; Hs 38.T; Hs 571.T; ES-2.

Prostate cancer: 22rv1; PC-3; DU-145.

Bladder/Urinary cancer: Hs 195.T; Hs 172.T; Hs 228.T; 5637; HT-1376; HT-1197; UM-UC-3; SW 780; J82; SCaBER; T24; TCCSUP; Hs 789.T; Hs 769.T.

Endometrial/Uterine cancer: KLE; HEC-1-A; HEC-1-B; RL95-2; SK-UT-1; SK-UT-IB; MES-SA; MES-SA/Dx5; MES-SA/MX2.

Stomach cancer: AGS; Hs 740.T; SNU-1.

Liver cancer: C3A; SNU-398; SNU-449; SNU-182; SNU-475; Hep 3B2.1-7; Hep G2; SNU-387; SNU-423.

Thyroid cancer: TT; SW579.

Bone cancer: Hs 819.T; SW 1353; Hs 900.T; Hs 903.T; Hs 919.T; 143.98.2; MG-63; HOS; KHOS/NP(R-970-5); KHOS-240S; KHOS-321H; MNNG/HOS (Cl #5); Hs 3.T; Hs 39.T; Hs 184.T; Hs 188.T; Hs 387.T; Hs 704.T; Hs 707(A).T; Hs 735.T; Hs 755(B).T; Hs 781.T; Hs 792(B).T; Hs 805.T; Hs 811.T; Hs 866.T; Hs 870.T; Hs 871.T; Hs 889.T; Hs 890.T; Murphy; R-970-5; TE 417.T; TE 418.T; TO 203.T; HT 728.T; Hs 14.T; T1-73; 143B; 143B PML BK TK; Saos-2; U-2-OS; SK-ES-1; Hs 822.T; Hs 863.T; R^{D}-ES; Hs 706.T; Hs 737.T; Hs 821.T; Hs 846.T; Hs 883.T; Hs 127.T; TE 76.T; TE 130.T.

Connective tissue cancer: TE 115.T; HT-1080; Hs 778(A).T; Hs 778(B).T; Hs 15.T; SW 684; Hs 93.T; Hs 5.T; SW 872; Hs 88.T; Hs 864.T; TE 441.T; TE 617.T; Hs 729.T; Hs 132.T; Hs 701.T.

Muscle cancer: TE 149.T; RD; A-673; Hs 729; A-204; Hs 94.T.

CNS cancer: CCF-STTG1; SW 1088; SW 1783; A172; U-138 MG; DBTRG-05MG; LN-18; LN-229; U-87 MG; U-118 MG; M059K; M059J; T98G; LNZTA3WT4; LNZTA3WT11; Hs 683; D341 Med; Daoy; CHP-212; IMR-32; H4; PFSK-1.

Melanoma: WM-115; Hs 600.T; Hs 688(A).T; Hs 839.T; Hs 852.T; Hs 906(A).T; Hs 906(B).T; Hs 908.5 k; Hs 936.T; Hs 936.T(C1); Hs 939.T; A101D; CHL-1; C32TG; C32; Hs 934.T; Hs 935.T; G-361; A-375; A375.S2; COLO 829; Hs 940.T; HT-144; Malme-3M; RPMI-7951; SK-MEL-5; SK-MEL-24; SK-MEL-28; SK-MEL-31.

Leukemia: TF-1; TF-1a; TF-1.CN5a.1; HEL 92.1.7; SUP-B15; CCRF-SB; 8E5; KG-1; KG-1a; TALL-104; MOLT-4; CCRF-CEM; CCRF-HSB-2; MOLT-3; CEM/C2; CEM/C1; Loucy; Reh; THP-1; AML-193; Kasumi-1; Kasumi-3; BDCM; Kasumi-6; HL-60; HL-60/MX2; HL-60/MX1; J.CaM1.6; J.RT3-T3.5; D1.1; J45.01; MV-4-11; Kasumi-4; KU812; KU812E; KU812F; MEG-01; Mo-B; Mo; SUP-T1; GDM-1; CESS.

Myeloma: U266B1; RPMI 8226; NCI-H929.

Lymphoma: RPMI 6666; 1A2; Hs 313.T; Hs 777.T; Hs 602; H9/HTLV-IIIB; HuT 78; HT 1417; JSC-1; BCP-1; 2B8; Daudi; NC-37; EB-3; Raji; Jiyoye; NAMALWA; HS-Sultan; CA46; GA-10; 20B8; HKB-11; 1G2; 2F7; EB1; Ramos (RA1); Ramos.2G6.4C10; H9; HH; MJ; Toledo; BC-1; BC-2; U-937; TUR; Hs 604.T; Hs 751.T; Hs 445; Hs 611.T; Hs 616.T; BC-3; DB; Hs 505.T; Hs 491.T; Hs 518.T.

Some assays (e.g., a colony forming assay) may use either established cell lines, or fresh tumor biopsies surgically removed from patients with cancer. Exposures of the cells to compounds of the present invention may be carried out under simulated physiological conditions of temperature, oxygen, and nutrient availability in the laboratory. The endpoints for these in vitro assays can involve colony formation, the reduction of a mitochondrial enzyme substrate (e.g., reduction of MTT to a blue formazan) by viable cells, the binding of a dye to cellular proteins (e.g., sulforhodamine B), the uptake of “vital” dyes that are excluded from cells with an intact cytoplasmic membrane, or the incorporation of radiolabeled nutrients into a proliferating (viable) cell. A more detailed description of in vitro assays for testing the antineoplastic activity of iminopyrrolidone thiol amino acid conjugates of the present invention can be found in Hersh et al., J Natl Cancer Inst. 84: 1238-1244 (1992); Salmon et al., J Natl Cancer Inst. 86: 228-230 (1994); Dvorakova et al., Blood 97: 3544-3551 (2001); and Dorr et al., Int J Gastrointest Cancer 36: 15-28.

In vivo antitumor assays are generally conducted after antineoplastic activity is identified through in vitro cytotoxic assays, and most commonly use rodents for assessing the antineoplastic characteristics of selected compounds. In vivo tumor studies are typically carried out using human tumor cell lines that are implanted subcutaneously in the animals. The tumors are generally allowed to grow to a predetermined size (e.g., 100-200 mg), and the growth, or lack thereof, is then assessed periodically during a period of treatment with the investigative compound. The in vivo antineoplastic activity of a selected compound can be evaluated as a function of tumor growth inhibition calculated from the tumor weights in animals treated with the investigative compound, and in an untreated control group. Tumor weights are typically estimated from measurements of the dimensions of the tumor, which are often implanted in the front flank of the animals. The tumor growth inhibition is calculated as follows: T/C (%)=(median tumor weight of the treated group (T)/median tumor weight of the control group (C))×100. In other assay methods, particularly for non-localized tumors, survival can be used as an endpoint and a comparison made between treated animals and an untreated control group. A more detailed description of in vivo assays for testing the antineoplastic activity of iminopyrrolidone thiol amino acid conjugates of the present invention can be found in Hersh et al., J Immunother 13: 77-83 (1993); Don et al., Invest New Drugs 13: 113-116 (1995); and Pourpak et al., Anticancer Drugs 17: 1179-1184 (2006).

### III. PHARMACEUTICAL COMPOSITIONS OF THE PRESENT INVENTION

Compounds in accordance with the present invention can be used in the preparation of pharmaceutical compositions or medicaments useful for the treatment of cancer. Embodiments of the present invention include pharmaceutical compositions comprising a pharmaceutically acceptable carrier and an iminopyrrolidone thiol amino acid conjugate compound as described hereinbefore and having the formula:

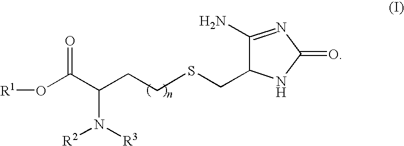

In Formula (I), R¹ is selected from hydrogen or substituted or unsubstituted (C₁-C₆) alkyl or (C₃-C₆) cycloalkyl, R² and R³ are independently selected from hydrogen, substituted or unsubstituted (C₁-C₆) alkyl, (C₄-C₆) cycloalkyl, or taken together with the N atom to which they are attached form a 3-6 membered heterocycloalkyl, and n is 1-5. In a preferred embodiment, a pharmaceutical composition of the present invention comprises a pharmaceutically acceptable carrier and a compound of Formula (I) in which R¹, R² and R³ are hydrogen, and n is 1.

Embodiments of pharmaceutical compositions in accordance with the present invention comprise a unit dose of an iminopyrrolidone thiol amino acid conjugate compound. In one embodiment, compounds of the present invention may be used in the preparation of a pharmaceutical composition useful for the treatment of malignant melanoma, multiple myeloma, lymphoma, prostate cancer, or pancreas cancer. In another embodiment, compounds of the present invention may be used in the preparation of a pharmaceutical composition useful for the treatment of cervical cancer, colorectal cancer, kidney cancer, lung cancer, non small-cell lung cancer, breast cancer, testicular cancer, ovarian cancer, bladder/urinary cancer, endometrial/uterine cancer, stomach cancer, liver cancer, thyroid cancer, bone cancer, connective tissue cancer, muscle cancer, central nervous system (CNS) cancer, or leukemia.

A. Dosage Forms

Pharmaceutical compositions of the present invention may be formulated for administration by any conventional means available for use in conjunction with pharmaceuticals, either as individual therapeutic agents or in combination with other therapeutic agents.

Iminopyrrolidone thiol amino acid conjugate compositions can be formulated in accordance with the teachings of the present invention in oral dosage forms such as tablets, capsules, pills, powders, granules, elixirs, tinctures, suspensions, syrups, or emulsions. Both tablets and capsules can be manufactured as immediate release products or as sustained release products to provide for an extended release over a period of hours, and may be enteric coated for selective disintegration in the patient's gastrointestinal tract. A composition comprising an iminopyrrolidone thiol amino acid conjugate can also be formulated in accordance with the present invention for administration in intravenous (bolus or infusion), intraperitoneal, subcutaneous, or intramuscular form, using any one of a number of dosage forms well known to those of ordinary skill in the pharmaceutical arts.

Pharmaceutical compositions of the present invention may, in various embodiments, comprise an iminopyrrolidone thiol amino acid conjugate in admixture with suitable pharmaceutical diluents, extenders, excipients, or carriers (collectively referred to herein as a pharmaceutically acceptable carrier) selected with respect to the intended form of administration and as consistent with conventional pharmaceutical practices. Pharmaceutically acceptable carriers may be combined with iminopyrrolidone thiol amino acid conjugate compounds of the present invention to produce either solid or liquid dosage forms.

Specific examples of pharmaceutically acceptable carriers that can be used to formulate oral dosage forms of the present invention are well known to one skilled in the art. See, for example, U.S. Pat. No. 3,903,297, which is incorporated herein by reference in its entirety for all purposes. Techniques and compositions for making dosage forms useful in the present invention are also well known to one skilled in the art. See, for example, 7 Modern Pharmaceutics, Chapters 9 and 10 (Banker & Rhodes, Eds., 1979); Pharmaceutical Dosage Forms: Tablets (Lieberman et al., 1981); Ansel, Introduction to Pharmaceutical Dosage Forms 2^{nd} Ed. (1976); Remington's Pharmaceutical Sciences, 17^{th} ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol. 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.), all of which are incorporated herein by reference in their entirety for all purposes.

Parenteral formulations of pharmaceutical compositions of the present invention may also include pharmaceutically acceptable carriers that make the formulations compatible with the type of injection or delivery system chosen. The production of sterile dosage forms for parenteral administration is well known in the pharmaceutical industry, and several sterilization processes are described in detail in the literature. In many cases, the stability of a chemotherapeutic agent in a parenteral formulation can be maximized by formulating the drug in a lyophilized dosage form. The lyophilization process may summarily be described as a “freeze-drying” process, in which a pharmaceutical agent is dissolved with or without excipients in a suitable solvent, and then sterilized by, for example, passing the bulk solution through a bacteria-retentive filter. Following sterilization, the solution is then filled into individual containers and frozen in a freeze-drying chamber. Finally, application of a vacuum to the freeze-drying chamber results in sublimation (primary drying) and desorption (secondary drying) of the solvent from the individual containers, which are then sealed to maintain sterility of the drug product. Pharmaceutical compositions of the present invention may comprise sterile or lyophilized dosage forms.

As will be appreciated, pharmaceutical compositions of the present invention may also be formulated in other dosage forms known to those skilled in the art. For example, compounds of the present invention may be formulated as liposome delivery systems, such as small unilamellar vesicles, large unilamellar vesicles, and multilamellar vesicles. Liposomes can be formed from a variety of phospholipids, such as cholesterol, stearylamine, or phosphatidylcholines. Compounds of the present invention may also be formulated via coupling to soluble polymers as targetable drug carriers or as a prodrug. Suitable soluble polymers include polyvinylpyrrolidone, pyran copolymer, polyhydroxylpropylmethaerylamide-phenol, polyhydroxyethylasparta-midephenol, and polyethyleneoxide-polylysine substituted with palmitoyl residues. Furthermore, compounds in accordance with the present invention can be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Useful pharmaceutical dosage forms for administration of the iminopyrrolidone thiol amino acid conjugates of the present invention include:

1. Capsules

A large number of unit capsules may be prepared by filling standard two-piece hard gelatin capsules each with powdered active ingredient (e.g., 10-500 mg) and one or more of the following excipients: lactose (e.g., 5-150 mg); cellulose (e.g., 5-50 mg); and magnesium stearate (e.g., 5-10 mg).

2. Soft Gelatin Capsules

A mixture of active ingredient in a digestible oil such as soybean oil, cottonseed oil, or olive oil may be prepared and injected by means of a positive displacement pump into gelatin to form soft gelatin capsules containing the active ingredient (e.g., 100-500 mg). The capsules may then be washed and dried.

3. Tablets

A large number of tablets may be prepared by conventional procedures so that the appropriate dosage unit of active ingredient (e.g., 100-500 mg) is included along with one or more of the following excipients: colloidal silicon dioxide (e.g., 0.1-1.0 mg); magnesium stearate (e.g., 2-5 mg); microcrystalline cellulose (e.g., 50-275 mg); starch (e.g., 5-15 mg); and lactose (e.g., 90-100 mg). Appropriate coatings may be applied to increase palatability of delay absorption.

4. Injectable Solution

A parenteral composition suitable for administration by injection may be prepared by stirring the appropriate amount of active ingredient (e.g., 1-2% by weight) in 10% by volume propylene glycol and water. The solution may be made isotonic with sodium chloride and sterilized.

5. Suspension

An aqueous suspension may be prepared for oral administration so that each 5 ml contain the appropriate amount of finely divided active ingredient (e.g., 100 mg) and one or more of the following excipients: sodium carboxymethyl cellulose (e.g. 200 mg); sodium benzoate (e.g., 5 mg); sorbitol solution (e.g., 1 mg); and vanillin (e.g., 0.025 mg).

B. Doses

The dosage forms described hereinbefore may contain from about 1.0 mg to about 5000 mg of active ingredient per unit. Doses of chemotherapeutic agents are conventionally denoted in units of milligrams per square meter (mg/m²) of body surface area (BSA). Typically, an adult human will have approximately 1.75 m² of BSA. The appropriate therapeutic dose for a particular patient may be calculated from the patient's BSA metric in conjunction with the particular disease being treated, and may be administered in one or more doses several times per day or per week. It will be appreciated that multiple dosage units may be required to deliver a therapeutically effective amount to the patient.

By way of general guidance, therapeutically effective doses of iminopyrrolidone thiol amino acid conjugates of the present invention may range from about 10 mg/m² to about 3500 mg/m², and preferably from about 150 mg/m² to about 2000 mg/m². Other preferred doses range from about 500 mg/m² to about 1500 mg/m².

Intravenously, the most preferred rates of administration can range from about 0.1 to about 10 mg/kg/minute during a constant rate infusion. Pharmaceutical compositions of the present invention can be administered as a single daily dose, or the total daily dose may be administered in divided doses of two, three, or four times daily.

C. Pharmaceutical Kits

Pharmaceutical compositions of the present invention may comprise a component of a pharmaceutical kit, in an embodiment. Such pharmaceutical kits may include a pharmaceutical composition comprising a therapeutically effective amount of an iminopyrrolidone thiol amino acid conjugate of the present invention and printed instructions for using the composition in the treatment of cancer. Pharmaceutical kits of the present invention can further include, if desired, one or more of various conventional pharmaceutical kit components, such as, for example, containers with one or more pharmaceutically acceptable carriers, additional containers, etc., as will be readily apparent to those skilled in the art. The printed instructions, either as inserts or as labels, may indicate quantities of the components to be administered, guidelines for administration, and/or guidelines for mixing the components. It should be understood that although the specified materials and conditions are important in practicing the invention, unspecified materials and conditions are not excluded so long as they do not prevent the benefits of the invention from being realized.

In one embodiment, the printed instructions may describe using the compound or composition of the present invention in the treatment of a cancer as described hereinafter with reference to methods of treating cancer patients.

### IV. METHODS OF TREATING CANCER PATIENTS

Drug products formulated according to the descriptions provided hereinbefore and embodying compounds and compositions of the present invention are useful in the treatment of a variety of cancers, including without limitation, solid tumors such as malignant melanoma, prostate cancer, pancreas cancer, cervical cancer, colorectal cancer, kidney cancer, lung cancer, non small-cell lung cancer, breast cancer, testicular cancer, ovarian cancer, bladder/urinary cancer, endometrial/uterine cancer, stomach cancer, liver cancer, thyroid cancer, bone cancer, connective tissue cancer, muscle cancer, and CNS cancer, as well as hematological malignancies such as multiple myeloma, lymphomas, and leukemias.

As will be appreciated, iminopyrrolidone thiol amino acid conjugate compounds and compositions in accordance with the teachings of the present invention may be used in the treatment of cancer either alone, or in combination with a second antineoplastic agent. As used herein, the terms “combination therapy” and “adjunct therapy” mean that a patient in need of the drug is treated with or given another drug for the disease in conjunction with an iminopyrrolidone thiol amino acid conjugate. In various embodiments, the combination therapy can be sequential therapy where the patient is treated first with one drug and then the other or the two drugs can be administered simultaneously.

Methods of treating cancer with iminopyrrolidone thiol amino acid conjugate compounds and compositions in accordance with the teachings of the present invention may comprise any suitable method that is effective in the treatment of the particular cancer or tumor type being treated. Treatment may be facilitated by oral, rectal, topical, or parenteral administration, or by injection into the tumor or cancer. It is believed that parenteral treatment by intravenous, subcutaneous, or intramuscular application of an iminopyrrolidone thiol amino acid conjugate compound, formulated with an appropriate pharmaceutically acceptable carrier to facilitate application, will be the preferred method of administering compounds and compositions of the present invention. The compounds and compositions of the present invention can be formulated in any suitable manner applicable to the chosen route of administration, as will be familiar to those skilled in the pharmaceutical art, and may be administered in a therapeutically effective dose, which may vary according to the particular disease being treated, the severity of the disease, and the response to treatment.

One skilled in the art will recognize that the efficacy of the compounds can be ascertained through routine screening using known cancer cell lines both in vitro and in vivo. Cell lines are available from American Tissue Type Culture or other laboratories as described hereinbefore.

A. Measuring Response to Pharmaceutical Formulations

Tumor load is generally assessed prior to therapy by means of objective scans of the tumor such as with x-ray radiographs, computerized tomography (CAT scans), nuclear magnetic resonance (NMR) scans or direct physical palpation of the tumor mass. Alternatively, the tumor may secrete a marker substance such as alphafetoprotein from colon cancer, CA125 antigen from ovarian cancer, or serum myeloma “M” protein from multiple myeloma. The levels of these secreted products then allow for an estimate of tumor burden to be calculated. These direct and indirect measures of the tumor load are done pretherapy, and are then repeated at intervals following the administration of the drug in order to gauge whether or not an objective response has been obtained. An objective response in cancer therapy generally indicates >50% shrinkage of the measurable tumor disease (a partial response), or complete disappearance of all measurable disease (a complete response). Typically these responses must be maintained for a certain time period, usually one month, to be classified as a true partial or complete response. In addition, there may be stabilization of the rapid growth of a tumor or there may be tumor shrinkage that is <50%, termed a minor response or stable disease. In general, increased survival is associated with obtaining a complete response to therapy, and in some cases, a partial response, if maintained for prolonged periods can also contribute to enhanced survival in the patient.

Patients receiving chemotherapy are also typically “staged” as to the extent of their disease before beginning chemotherapy, and are then restaged following chemotherapy to see if this disease extent has changed. In some situations the tumor may shrink sufficiently, and if no metastases are present, to make surgical excision possible after chemotherapy treatment where it was not possible beforehand due to the widespread disease. In this case the chemotherapy treatment with the novel pharmaceutical compositions is being used as an adjuvant to potentially curative surgery. In addition, patients may have individual lesions in the spine or elsewhere that produce symptomatic problems such as pain and these may need to have local radiotherapy applied. This may be done in addition to the continued use of the systemic pharmaceutical compositions of the present invention.

B. Assessing Toxicity and Setting Dosing Regimens

Patients are assessed for toxicity with each course of chemotherapy, typically looking at effects on liver function enzymes and renal function enzymes such as creatinine clearance or BUN as well as effects on the bone marrow, typically a suppression of granulocytes important for fighting infection and/or a suppression of platelets important for hemostasis or stopping blood flow. For such myelosuppressive drugs, the nadir in these normal blood counts is reached between 1-3 weeks after therapy and recovery then ensues over the next 1-2 weeks. Based on the recovery of normal white blood counts, treatments may then be resumed.

In general, complete and partial responses are associated with at least a 1-2 log reduction in the number of tumor cells (a 90-99% effective therapy). Patients with advanced cancer will typically have >10⁹ tumor cells at diagnosis, and multiple treatments will often be required in order to reduce tumor burden to a very low state and potentially obtain a cure of the disease.

Treatment schedules or dosing regimens for the administration of compounds or pharmaceutical compositions in accordance with the present invention conventionally comprise cycles of treatment wherein a specified dose of the compound, or each composition of a combination therapy, is administered to a patient at defined intervals over the period of a cycle, and then repeated in each subsequent cycle. The period of a cycle may be defined in any suitable manner, and may comprise, for example, a twenty-one day cycle, a twenty-eight day cycle, or the like. Within the period of a cycle of treatment, the specified dose of a compound in accordance with the present invention can be administered to the patient at defined intervals, such as for example, for five consecutive days every other week (e.g., days 1-5 and 15-19 of a 28-day cycle), for five consecutive days every three weeks (e.g., days 1-5 of a 21-day cycle), once per week (e.g., days 1, 8 and 15 of a 21-day cycle), or the like.

C. Clinical Management of Patients

At the end of a treatment cycle with a pharmaceutical composition of the present invention, which could comprise several weeks of continuous drug dosing, patients will be evaluated for response to therapy (complete and partial remissions), toxicity measured by blood work and general well-being classified performance status or quality of life analysis. The latter includes the general activity level of the patient and their ability to do normal daily functions. It has been found to be a strong predictor of response and some anticancer drugs may actually improve performance status and a general sense of well-being without causing significant tumor shrinkage. The antimetabolite gemcitabine is an example of such a drug that was approved in pancreatic cancer for benefiting quality of life without changing overall survival or producing a high objective response rate. Thus, for some cancers that are not curable, the pharmaceutical formulations may similarly provide a significant benefit, well-being performance status, etc. without affecting true complete or partial remission of the disease.

In hematologic disorders such as multiple myeloma, lymphoma and leukemia, responses are not assessed via the measurement of tumor diameter since these diseases are widely metastatic throughout the lymphatic and hematogenous areas of the body. Thus, responses to these diffusely disseminated diseases are usually measured in terms of bone marrow biopsy results wherein the number of abnormal tumor cell blasts are quantitated and complete responses are indicated by the lack of detection (e.g., microscopic detection) of any tumor cells in a bone marrow biopsy specimen. With the B-cell neoplasm multiple myeloma, a serum marker, the M protein, can be measured by electrophoresis and, if substantially decreased, this is evidence of the response of the primary tumor. Again, in multiple myeloma, bone marrow biopsies can be used to quantitate the number of abnormal tumor plasma cells present in the specimen. For these diseases, higher dose therapy is typically used to affect responses in the bone marrow and/or lymphatic compartments.

While the invention is described here in the context of a limited number of embodiments, and with reference to specific details and examples, the invention may be embodied in many forms without departing from the spirit of the essential characteristics of the invention. The exemplary and described embodiments, including what is described in the summary of the invention and the abstract, are therefore to be considered in all respects as illustrative and not restrictive. The scope of the invention is indicated by the appended claims rather than by the foregoing description, and all changes that come within the meaning and range of equivalency of the claims are intended to be embraced therein.

### EXAMPLES

The following examples are offered to illustrate, but not to limit, the claimed invention.

### Materials

Human malignant melanoma A375 cells, human prostate cancer PC-3 and DU-145 cells, human myeloma 8226/s and 8226/110 cells, and human pancreatic cancer MiaPaCa cells were obtained from the American Type Culture Collection (Manassas, Va.). DB and Raji lymphoma cells were kindly provided by Dr. Alan List (University of Arizona, Tucson, Ariz.). All cell lines were cultured in RPMI 1640 media (Mediatech, Herndon, Va.) enhanced with 5% (v/v) (10% for DB and Raji cells) heat-inactivated bovine calf serum (Hyclone Laboratories, Logan, Utah), 2 mM L-glutamine, 100 U/ml penicillin, and 100 μg/ml streptomycin (each from Invitrogen, Carlsbad, Calif.) in a humidified incubator containing 5% CO₂ at 37° C.

Imexon was obtained as a generous donation from AmpliMed Corporation (Tucson, Ariz.), and L-simexonyl homocysteine was prepared according to the exemplary synthesis route described herein from L-homocysteine thiolactone (Sigma Chemical Co., St. Louis, Mo.). MTT and SRB assay dyes were obtained from Sigma Chemical Co., and the annexin V—FITC/PI and 1× binding buffer were obtained from BioVision Labs (Mountain View, Calif.).

### Example 1

Example 1 illustrates a method of determining the antineoplastic activity of iminopyrrolidone thiol amino acid conjugates in vitro. Ninety-six (96) well plates (BD Biosciences, San Jose, Calif.) were seeded with approximately 5000 cells in 160 μl of growth medium per well in the last eleven columns of each plate. The first column of each plate was filled with 160 μl of growth medium containing no cells to be used as a blank. Following a 24-hour incubation period, the cells in the last ten columns were drugged (leaving row one as a blank and row two as a control with uninhibited cell growth) with 40 μl of either simexonyl homocysteine or imexon in growth medium. The quantity of drug added to the wells of each respective column was constant, but varied from column to column such that the resulting concentration in the total volume of each individual well ranged from 1 μM to 1000 μM. Three or four days (the specific timing is indicated in the associated figures and Table 1) after the addition of either simexonyl homocysteine or imexon to the cells, the plates containing 8226/s, 8226/110, DB, or Raji cells were analyzed using the MTT assay (see, e.g., Mosmann, T., J Immunol Met. 65: 55-63 (1983); Rubinstein et al., J Natl Cancer Inst. 82: 1113-1118 (1990)), while plates containing A375, PC-3, DU-145, and MiaPaCa cells were analyzed using the SRB assay (see, e.g., Skehan et al., J Natl Cancer Inst. 82: 1107-1112 (1990)).

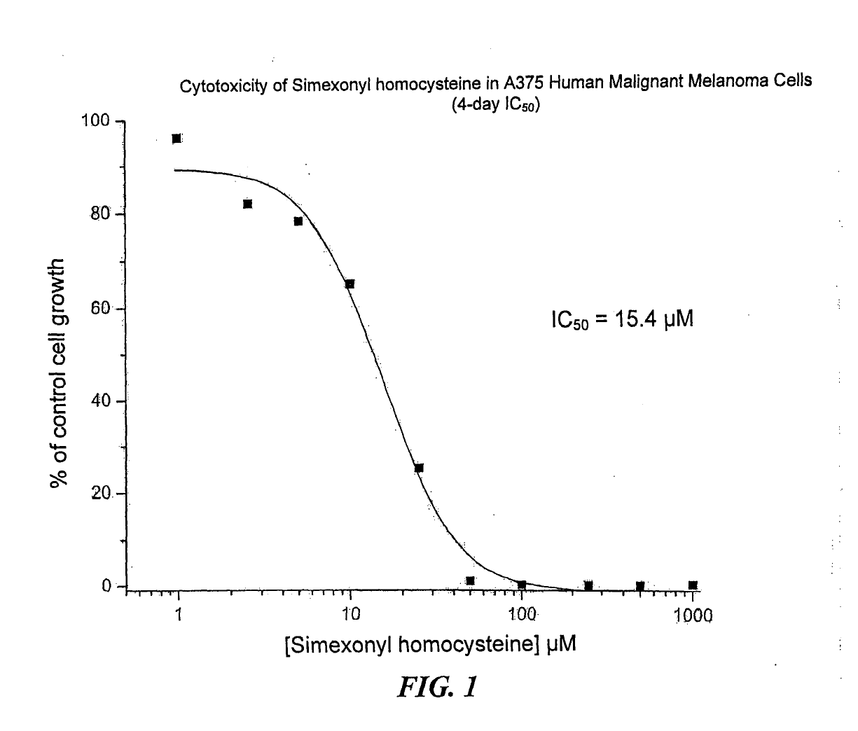

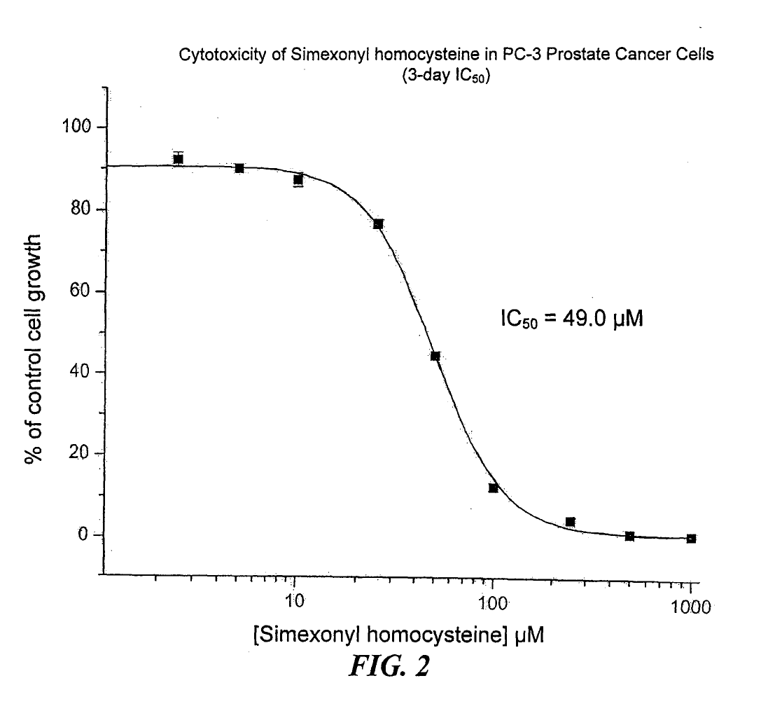

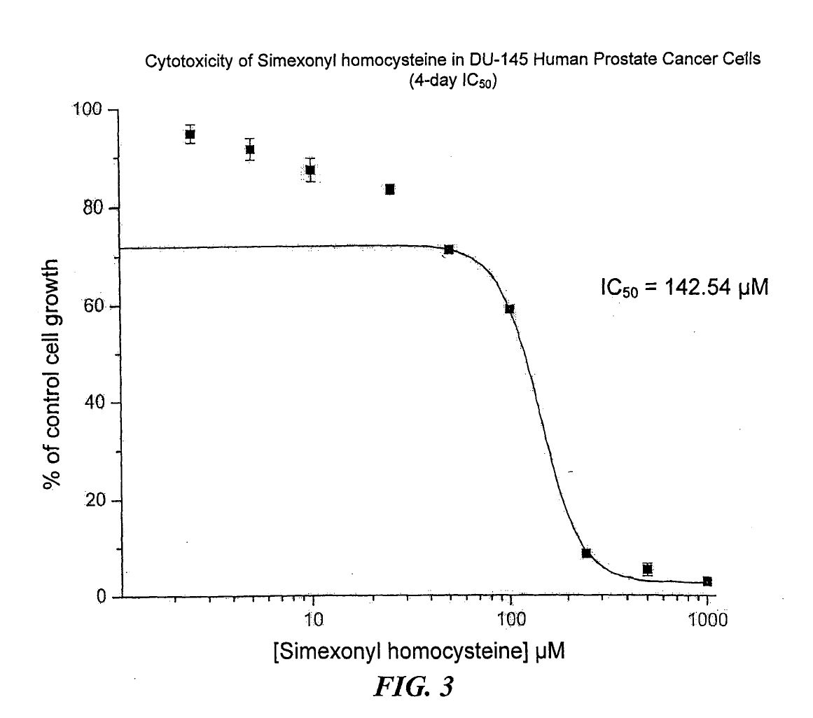

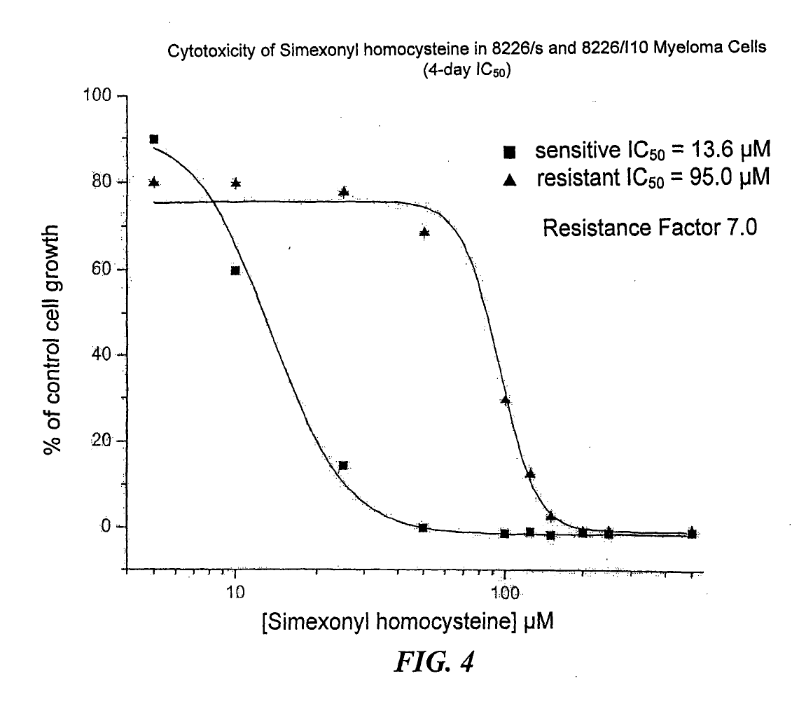

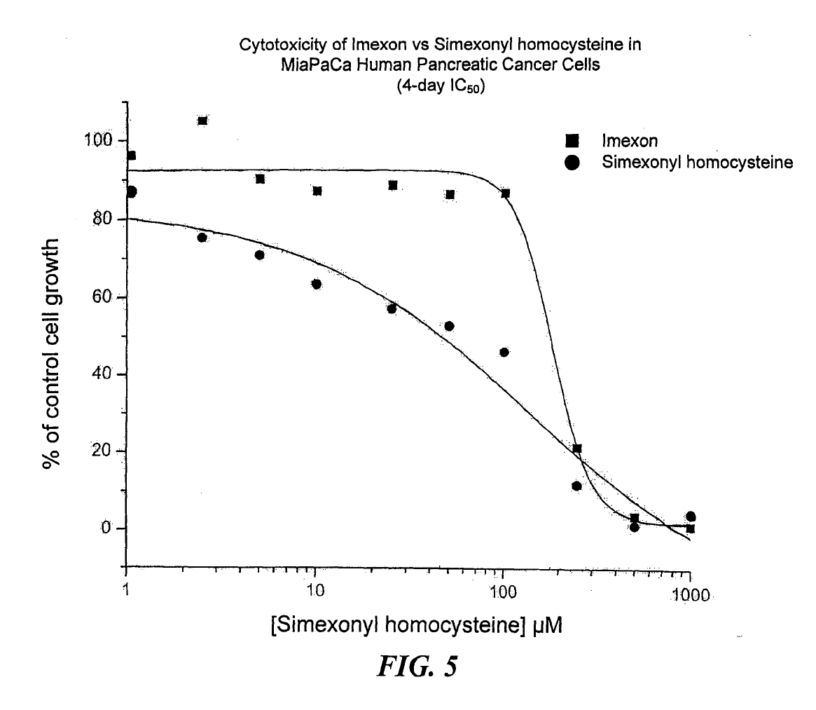

The cytotoxicity of simexonyl homocysteine in A375 human malignant melanoma, PC-3 and DU-145 human prostate cancer, 8226/s and 8226/110 human myeloma, and in comparison to the cytotoxic activity of imexon in MiaPaCa human pancreatic cancer cells are illustrated as a percentage of control cell growth as a function of drug concentration in FIGS. 1-5. The IC₅₀ is the drug concentration required to achieve 50% growth inhibition.

Table 1 shows the numerical results of these cytotoxicity studies.

TABLE 1
Results of Cytotoxicity Studies

| | | IC₅₀ at 72* or |
|---|---|---|
| Drug | Cell Line | 96 hours (μM) |
| simexonyl homocysteine | A375 - Melanoma | 15.4 |
| simexonyl homocysteine | PC-3 - Prostate | 49.0* |
| simexonyl homocysteine | DU-145 - Prostate | 142.54 |
| simexonyl homocysteine | 8226/s - Myeloma | 13.6 |
| simexonyl homocysteine | 8226/I10 - Myeloma | 95.0 |
| simexonyl homocysteine | MiaPaCa - Pancreas | 46 |
| imexon | MiaPaCa - Pancreas | 190 |
| simexonyl homocysteine | DB - Lymphoma | 23 |
| imexon | DB - Lymphoma | 54 |
| simexonyl homocysteine | Raji - Lymphoma | 22 |
| imexon | Raji - Lymphoma | 50 |

As illustrated in the figures and in Table 1, simexonyl homocysteine shows cytotoxic activity in a variety of tumor cell lines in vitro. Moreover, the results of the cytotoxicity assays in which the antineoplastic activity of simexonyl homocysteine is directly compared to that of imexon, a compound shown to have substantial anticancer activity in human clinical studies, indicates that the iminopyrrolidone thiol amino acid conjugates of the present invention hold significant promise as effective therapies in the treatment of cancer.

### Example 2

Example 2 illustrates a method of measuring apoptosis induced by iminopyrrolidone thiol amino acid conjugates in vitro. DB lymphoma cells were cultured in ninety-six (96) well plates (BD Biosciences) under conditions as described hereinbefore. Following a 24-hour incubation period, the cells were drugged (or left undrugged) with simexonyl homocysteine in growth medium to yield a drug concentration of either 0 μM, 45 μM, 90 μM, or 180 μM in each well, respectively. Forty eight hours after drug exposure, the percentage of necrotic, viable, proapoptotic, and apoptotic cells in each respective well was measured using an annexin V—FITC (fluorescein isothiocyanate labeled annexin V) apoptosis detection kit (BioVision Labs) according to the manufacturer's instructions in conjunction with a Bectin Dickenson FACScan (San Jose, Calif.) fluorescence activated cell sorter using CellQuest Pro software (BD Biosciences). The manufacturer's instructions comprise: (i) the collection of 1-5×10⁵ cells by centrifugation; (ii) resuspension of the cells in 500 μl of 1× binding buffer (HEPES, NaCl, CaCl₂ at pH 7.4); (iii) the addition of 5 μl of annexin V—FITC and 5 μl of propidium iodide; (iv) incubation of the cell assay at room temperature for five minutes in the dark; and (v) quantification by flow cytometry. A more detailed discussion of the apoptosis assay can be found in Koopman et al., Blood 84: 1415-1420 (1994).

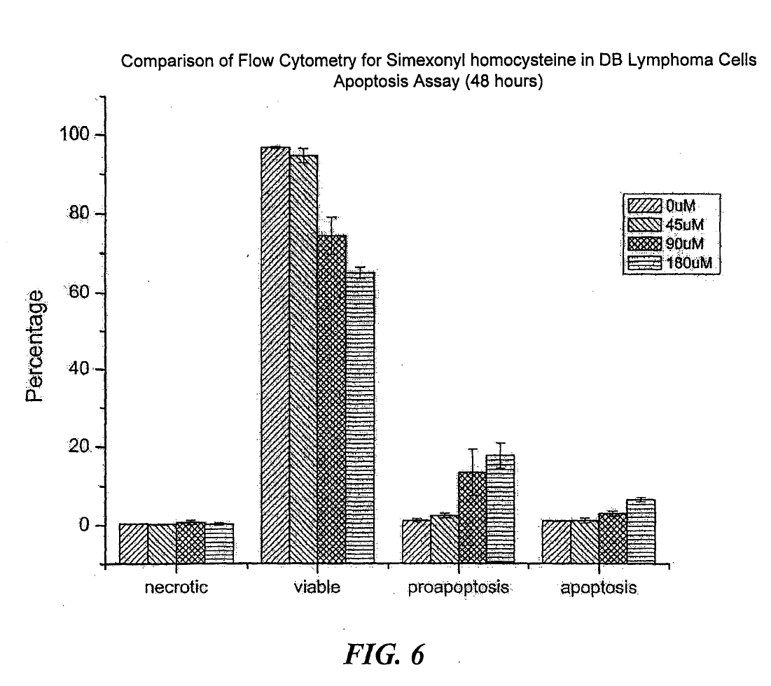

The results of the apoptosis assay, at each of the respective drug concentrations, are illustrated in FIG. 6. As can be seen in the figure, increasing concentrations of simexonyl homocysteine induced apoptosis in the DB lymphoma cells, as shown by the increasing percentage of proapoptotic and apoptotic cells at higher drug concentrations. A threshold intensity for the fluorescence signal permits one to distinguish between proapoptotic and apoptotic cells in the flow cytometric analysis, but both are indicative of an induction of apoptosis by the drug.

### Example 3

Example 3 illustrates a method of synthesizing, purifying, and characterizing L-simexonyl homocysteine.

L-homocysteine thiolactone (2 mM, 307.2 mg) was dissolved in 2 ml of 5 N NaOH under argon. The mixture was allowed to react for 30 minutes at 37° C., and was then titrated to pH 7.5 with 12 N, 6 N, and 1 N HCl. Imexon (2 mM, 222 mg) was added immediately and the mixture was allowed to react for 5 hours at 37° C. while being monitored by HPLC, and was then stopped by freezing at −20° C. (5 hours was identified as the duration which yielded the maximum product concentration).

The L-simexonyl homocysteine produced by the foregoing synthesis scheme was purified by semi-preparative HPLC to remove unreacted starting materials and to desalinate the product of further characterization. A C18 (10μ, 250×10 mm) semi-preparative column with a 100% water mobile phase was used for the purification. The L-simexonyl homocysteine was collected by hand following identification by its relative retention time (˜6.4 minutes) and characteristic absorbance by continuous diode array UV scan (˜200 nm).

The purified L-simexonyl homocysteine was characterized by mass spectrometry, giving a mass of 246.9 by ESI+ on a Finnigan LCQ HPLC/MS instrument, compared to the theoretical molecular weight of 246.3.

What is claimed is:

1. A compound having the formula: 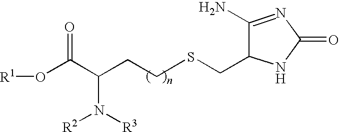
wherein
R¹ is selected from hydrogen or substituted or unsubstituted (C₁-C₆) alkyl or (C₃-C₆) cycloalkyl,
R² and R³ are independently selected from hydrogen, substituted or unsubstituted (C₁-C₆) alkyl, (C₄-C₆) cycloalkyl, or taken together with the N atom to which they are attached form a 3-6 membered heterocycloalkyl, and
n is 1-5.

2. The compound of claim 1, wherein n is 1-3.

3. The compound of claim 1, wherein R¹ is hydrogen and n is 1.

4. The compound of claim 1, wherein R² and R³ are hydrogen and n is 1.

5. The compound of claim 1, wherein R¹ is (C₁-C₃) alkyl.

6. The compound of claim 1, wherein R¹, R², and R³ are hydrogen and n is 1.

7. A pharmaceutical composition comprising a unit dose of a compound having the formula: 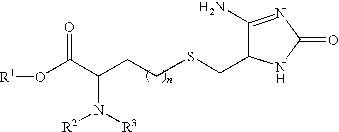
wherein
R¹ is selected from hydrogen or substituted or unsubstituted (C₁-C₆) alkyl or (C₃-C₆) cycloalkyl,
R² and R³ are independently selected from hydrogen, substituted or unsubstituted (C₁-C₆) alkyl, (C₄-C₆) cycloalkyl, or taken together with the N atom to which they are attached form a 3-6 membered heterocycloalkyl, and
n is 1-5,
or a pharmaceutically acceptable salt thereof; and
a pharmaceutically acceptable carrier.

8. The pharmaceutical composition of claim 7, wherein R¹, R², and R³ are hydrogen and n is 1.

9. The pharmaceutical composition of claim 7 in sterile dosage form.

10. The pharmaceutical composition of claim 7, wherein the pharmaceutically acceptable salt is selected from a group consisting of chlorides, phosphates, sulfates, tosylates, benzoylates, and mesylates.

11. A method of treating cancer comprising administering to a patient in need thereof a therapeutically effective amount of a composition comprising a compound of the formula: 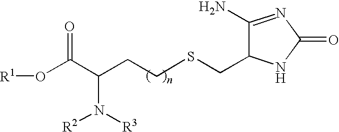
wherein
R¹ is selected from hydrogen or substituted or unsubstituted (C₁-C₆) alkyl or (C₃-C₆) cycloalkyl,
R² and R³ are independently selected from hydrogen, substituted or unsubstituted (C₁-C₆) alkyl, (C₄-C₆) cycloalkyl, or taken together with the N atom to which they are attached form a 3-6 membered heterocycloalkyl, and
n is 1-5.

12. The method of claim 11, wherein n is 1-3.

13. The method of claim 11, wherein R¹ is hydrogen and n is 1.

14. The method of claim 11, wherein R² and R³ are hydrogen and n is 1.

15. The method of claim 11, wherein R¹, R², and R³ are hydrogen and n is 1.

16. The method of claim 11, wherein the cancer is selected from a group consisting of malignant melanoma, multiple myeloma, lymphoma, prostate cancer, and pancreas cancer.

17. The method of claim 11, wherein the cancer is selected from cervical cancer, colorectal cancer, kidney cancer, lung cancer, non small-cell lung cancer, breast cancer, testicular cancer, ovarian cancer, bladder/urinary cancer, endometrial/uterine cancer, stomach cancer, liver cancer, thyroid cancer, bone cancer, connective tissue cancer, muscle cancer, CNS cancer, and leukemia.

18. The method of claim 11, wherein the composition comprises a solid dosage form for oral administration to the patient.

19. A compound having the formula:

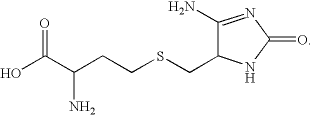

20. A method of treating cancer comprising administering to a patient in need thereof a therapeutically effective amount of a compound of the formula:

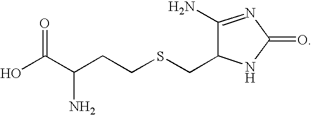

21. The method of claim 20, wherein the cancer is selected from pancreas cancer, prostate cancer, melanoma, multiple myeloma or lymphoma.
